(19) United States
(12) Patent Application Publication
(10) Pub. No.: US 2006/0211974 A1
(43) Pub. Date: Sep. 21, 2006

(54) Support for edematous scrotum

(76) Inventors: Denise Bland, Weslaco, TX (US); Renee Michelle St. Laurent, Houston, TX (US)

Correspondence Address: LOCKE LIDDELL & SAPP LLP, 600 TRAVIS, 3400 CHASE TOWER, HOUSTON, TX, 77002-3095, US

(21) Appl. No.: 11/242,241

(22) Filed: Oct. 3, 2005

Related U.S. Application Data
(60) Provisional application No. 60/619,377, filed on Oct. 15, 2004.
(60) Provisional application No. 60/697,112, filed on Jul. 7, 2005.

(51) Int. Cl. A61F 13/00

(52) U.S. Cl. 602/67

(57) ABSTRACT

A support device for an edematous scrotum is provided, which comprises a body portion adapted to be worn about a man's mid-section, a first connector for joining ends of the body portion together, and a plurality of second connectors. A receptacle is also provided and is adapted to receive and support an edematous scrotum and comprises a plurality of support members each adapted to couple with one of the plurality of second connectors to thereby provide comforting support to the scrotum.

### CROSS REFERENCE TO RELATED APPLICATIONS

This application claims benefit of U.S. provisional application No. 60/619,377, filed on Oct. 15, 2004.

### STATEMENT REGARDING FEDERALLY SPONSORED RESEARCH OR DEVELOPMENT

Not applicable.

### REFERENCE TO APPENDIX

Not applicable.

### BACKGROUND OF THE INVENTION

1. Field of the Invention

This disclosure relates generally to a device for providing support for an edematous scrotum, and more particularly to an undergarment for relieving pain associated with ambulating with an edematous scrotum.

2. Description of the Related Art

A variety of conditions and diseases may cause the male scrotum to swell or enlarge to an abnormal size. Such enlarged or swollen scrota are usually very painful and can render simple activities like walking unbearable. In addition, the stress caused by the edematous scrotum on surrounding tissues and structures may cause further damage, such as tearing of the skin or scrotal sac. While numerous scrotal support devices exist, none seem to address the specific problems associated with scrotal edema.

U.S. Pat. No. 6,245,036 is entitled “Male Genitalia Support Garment” and issued on Jun. 12, 2001. This patent discloses a male genitalia support garment that has a posterior testicular strap portion extending from a waistband portion of the garment. The posterior testicular strap portion is designed to be positioned behind the scrotum and the testicles to provide support to the wearer's scrotal contents. The posterior testicular strap portion is made of a generally unyielding material in the dynamic state so that a constant, non-variable amount of support is provided to the wearer.

U.S. Pat. No. Des. 304,775 is entitled “Scrotal Supporter” and issued on Nov. 28, 1989. This patent discloses an ornamental design for a scrotal supporter as shown in the 6 figures.

Other related arts include U.S. Pat. No. 1,208,758 entitled “Suspensory;” U.S. Pat. No. 3,225,761 entitled “Fatigue Support for Men No Leg Straps;” and U.S. Pat. No. 5,524,298 entitled “Male Genitals Garment.”

This application for patent discloses an improved support device for an enlarged, swollen or edematous scrotum to relieve the pain associated with such condition, especially during ambulation.

### BRIEF SUMMARY OF THE INVENTION

A support device for an enlarged scrotum is provided, comprising a body portion comprising a band of material adapted to be worn about a man's mid-section, a first connector for joining ends of the body portion together, and a plurality of second connectors. A receptacle is also provided and is adapted to receive and support an edematous scrotum comprising a plurality of support members each adapted to couple with one of the plurality of second connectors to thereby provide comforting support to the scrotum.

### BRIEF DESCRIPTION OF THE SEVERAL VIEWS OF THE DRAWINGS

FIG. 1 illustrates a support device for an enlarged scrotum according to the present invention.

FIG. 2a illustrates a top view of the inside of a receptacle according to the present invention.

FIG. 2b illustrates a front view of a receptacle according to the present invention.

FIG. 3 illustrates additional embodiments of a support device for an enlarged scrotum according to the present invention.

While the inventions disclosed herein are susceptible to various modifications and alternative forms, only a few specific embodiments are shown by way of example in the drawings and described in detail below. The figures and detailed descriptions of these specific embodiments are not intended to limit the breadth or scope of the inventive concepts or the appended claims in any manner. Rather, the figures and detailed written descriptions are provided to illustrate the inventive concepts to a person of ordinary skill in the art as required by 35 U.S.C. § 112.

### DETAILED DESCRIPTION

One or more illustrative embodiments incorporating the invention disclosed herein are presented below. Not all features of an actual implementation are described or shown in this application for the sake of clarity. It is understood that the development of an actual embodiment incorporating the present invention, numerous implementation-specific decisions must be made to achieve the developer's goals, such as compliance with system-related, business-related, government-related, and/or other constraints, which vary by implementation and from time to time. While a developer's efforts might be complex and time-consuming, such efforts would be, nevertheless, a routine undertaking for those of ordinary skill the art having benefit of this disclosure.

In general terms, Applicants have created a support device, such as an undergarment worn by a man, for imparting support to an enlarged, swollen or edematous scrotum, thereby relieving some of the pain associated with that condition. In broad terms, the device comprises a receptacle adapted to receive and support the enlarged scrotum. The receptacle is coupled, and preferably removably coupled, to a body portion that reacts the weight of the enlarged scrotum against the patient's body, such as the waist or hips. The receptacle coupling is preferably adjustable to provide the maximum level of comfort to the patient during ambulation or other activities.

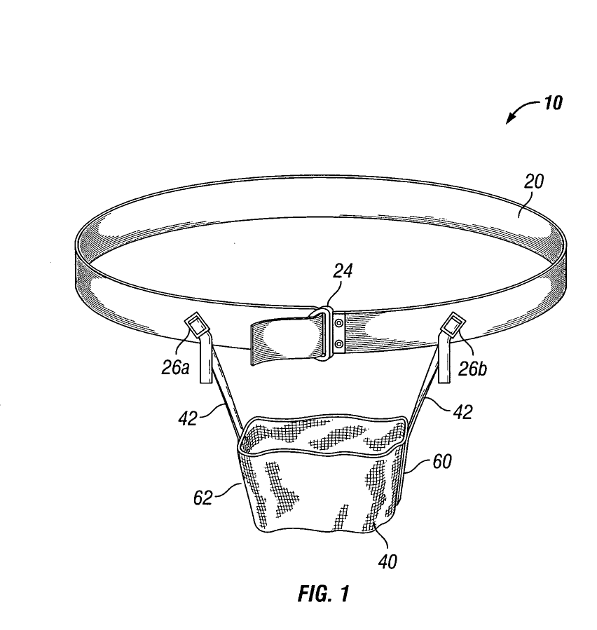

In more specific terms, FIG. 1 illustrates a first embodiment of a support device 10. The device 10 may comprise a body portion 20 and a receptacle 40. The receptacle 40 is adapted to receive and support all or substantially all of the enlarged scrotum (not shown). The receptacle 40 may comprise one or more members 42 for coupling the receptacle 40 to the body portion 20. In the embodiment shown in FIG. 1, two members 42 are shown disposed about opposite sides 60, 62 of the receptacle 40. The structures 42 may take the form of a strap or web, preferably made of fabric. In a preferred embodiment, the members 42 are fixedly and non-removably attached to the receptacle 40 at pre-determined locations, such as, for example, opposite sides of the receptacle 40. Alternately, the members 42 may be attached at the back portion or front portion of receptacle 40.

The body portion 20 may comprise a strap or web, preferably of fabric. The body portion 20 is adapted to be worn about the patient's mid-section, such as the hips or waist. The body portion 20 preferably may comprise a first connector 24 for removably fixing the body portion 20 in a type of circle or oval about the patient. The connector 24 may take a variety of forms, such as, but not limited to, a buckle ring, Velcro, snaps, buttons and other such connectors well known in the art. Alternately, the body portion may comprise an elastic band or ring without a connector. However, for some patients with edematous scrota, stepping into and out of a support device without a body portion connector 24 may be painful and/or cumbersome.

The body portion 20 also comprises a second set of connectors 26 for coupling the receptacle members 42 to the body portion 22. The body portion 20 is preferably sufficiently wide to carry the weight of the scrotum without digging in to the patient's waist. A presently preferred embodiment utilizes a 2″ wide body portion. Alternately, or additionally, other structures, such as, but not limited to, lamb's wool pads may be incorporated to reduce into body portion 20 to reduce any “hot” spots on the patient's body caused by wearing the device 10. In addition, the second set of connectors 26 may include support pieces, such as, but not limited to, semi-hard plastic, to back up the body portion 20 and prevent it from buckling under the weight of the enlarged scrotum.

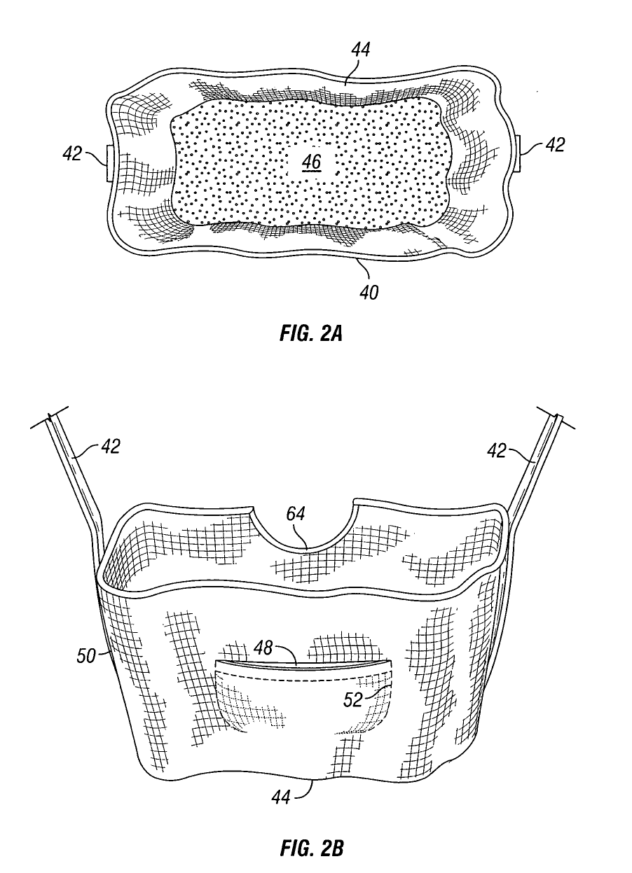

FIGS. 2a and 2b illustrate an embodiment of a receptacle 40 suitable for use with the present invention. FIG. 2a illustrates a top view looking into the scrotum receiving area of the receptacle 40. In a preferred embodiment, the bottom or floor 44 of the receptacle 40 may comprise one or more inserts 46 of foam or other soft padding or comfortable material. The insert 46 may be fixedly secured to the receptacle floor 44 or it may be removably affixed thereto. The insert 46 may also comprise a sanitary napkin or other removable sanitary liner, or a cooling insert, such as an ice pack or chemical cool pack.

FIG. 2b illustrates a front view of a receptacle 40 suitable for use with the present invention. Shown in FIG. 2b is an opening 48 in the front receptacle wall 50. The opening 48 provides access to an internal pouch 52 is shown for the receipt of cooling devices, such as ice packs or chemical cooling packs. It will be appreciated that one or more internal pouches 52 may be place about the receptacle 40 to provide any desired level of cooling relief. Also shown in FIG. 2b is a cutout or relief portion 64, which may be used to receive the base portion of the penis. In the configuration shown, the cutout portion 64 is placed on the backside of the receptacle 40 nearest the wearer's body. It will also be appreciated that a similar cutout portion (not shown) may be located on the front or outer most portion of the receptacle 40 to facilitate urination. A receptacle 40 for use with present invention may include both front and back cut out portions 64, either or none.

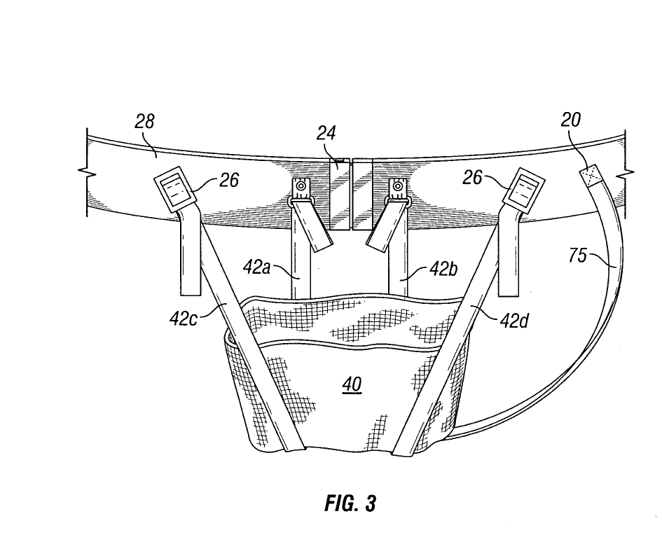

FIG. 3 illustrates additional embodiment of the present invention 10. In these embodiments, the receptacle 40 comprises four members 42a-d. Members 42a and 42b are preferably disposed on the posterior portion of the receptacle 40 adjacent the patient's groin region. The structures 42a and 42b may be placed on either side of the penis to provide support to the posterior portion of the receptacle 40. The structures 42c and 42d may be disposed on the side portions of the receptacle 40 (as shown in FIG. 1) or more toward the anterior portion as shown in FIG. 3 to provide additional support. The members 42c and 42d may be coupled to the body portion 20 by one or more of the connectors 26 discussed above. Likewise, the members 42a and 42b may be coupled to the body portion 20 by one or more of the connectors 26 discussed above, or more preferably, they may be coupled to the body portion 20 by adjustable snaps affixed to the outer body portion surface 28.

Also shown in FIG. 3 is a leg strap 75 configured to be worn between the legs and around the backside of the leg to provide additional support to the device 10. While only one leg strap 75 is shown in FIG. 3, it will be appreciated that another leg strap 75 for the other leg is contemplated as well. As discussed above with respect to the body portion 20, some wearers with enlarged scrota will prefer not to have to step into and out of a support device 10 with leg straps 75. So, while the present invention contemplates the use of leg straps in certain applications, the presently preferred embodiment does not require the wearer to step into or out of the device 10.

While it is presently preferred that all members 42 and straps 75 be removably affixed and/or adjustable to the body portion 20, it is also within the scope of this invention for one or more of the members or straps to be rigidly secured thereto. For example, the members 42a and 42b may be fixedly and non-adjustably secured to the body portion 20. It is preferred in this situation not to fixedly secure the anterior members 42c and 42d because to do so may diminish the ease of putting on the device.

The first connector 24 is shown in FIG. 3 to be midway between the members 42a and 42b, but it will be appreciated that the first connector 24 may be disposed at any convenient location on the body portion 20, such as at the back of the patient or on one side.

In use, it is contemplated that a patient will first place the body portion about his mid section in a location, such as hips or waist that is comfortable to the patient. Next, and depending upon the embodiment of the receptacle 40 chosen, the patient may affix any posterior receptacle members 42 to the body portion 20 and place the scrotum within the receptacle 40. The patient will then affix any other receptacle members 42 to the body portion 20, such as anterior members 42c and 42d. The patient may then adjust the members as desired to achieve the maximum level of comfort for the activity at hand, such as walking or riding in a car. Again, depending upon the embodiment chosen, the patient may utilize one or more cooling devices such as ice pack or chemical cooling packs to achieve desired level of comfort.

It will be appreciated that by making the body portion 20 and the receptacle 40 as separable components, a patient may have a plurality of receptacles 40 so that one may be cleaned while another is being worn. Because enlarged, swollen or edematous scrota are typically very sensitive and painful, it is preferred that the receptacle 40 be made out of soft breathable fabric or material. Because enlarged, swollen or edematous scrota are usually large, such as the size of an average honeydew melon, and heavy, it may be beneficial to provide more rigid structures to the receptacle 40, such as plastic bracing or panels. Varying combinations of structural support and comfortable materials may be used to accommodate scrota of varying sizes and conditions.

The invention has been described in the context of preferred and other embodiments and not every embodiment of the invention has been described. Obvious modifications and alterations to the described embodiments are available to those of ordinary skill in the art. The disclosed and undisclosed embodiments are not intended to limit or restrict the scope or applicability of the invention conceived of by the Applicants, but rather, in conformity with the patent laws, Applicants intends to protect all such modifications and improvements to the full extent that such falls within the scope or range of equivalent of the following claims.

1. A support device for an edematous scrotum comprising:
a body portion comprising a belt of material adapted to be worn about a man's mid-section, a first connector for joining ends of the body portion together, and a plurality of second connectors;
a receptacle adapted to receive and support an edematous scrotum comprising a plurality of support members each adapted to couple with one of the plurality of second connectors to thereby provide comforting support to the scrotum.

2. The device of claim 1, wherein the body portion comprises a fabric belt.

3. The device of claim 2, further comprising friction and/or pressure reducing pads on an inside surface of the belt adjacent the wearer's skin.

4. The device of claim 1, wherein the first connector is selected from the group consisting of: a ring buckle, Velcro, snaps, buttons and any combination of the foregoing.

5. The device of claim 1, wherein the receptacle is made from a soft fabric suitable for supporting an edematous scrotum.

6. The device of claim 1, further comprising an insert in the receptacle for cushioning and/or cooling the scrotum.

7. The device of claim 5, wherein the insert is removable.

8. The device of claim 1, further comprising one or more pouches in the wall of the receptacle for receiving scrotum-cooling products.

9. The device of claim 1, wherein the plurality of support members consists of two support members located substantially opposite each other.

10. The device of claim 1, wherein the plurality of support members consists of four supports, two of which are located on a posterior portion of the receptacle on either side of the penis, the remaining two of which are located adjacent and anterior portion of the receptacle.

11. The device of claim 9, wherein the posterior support members are rigidly fixed to the body portion and are not adjustable, and the two support members adjacent the anterior portion is removably fixed and adjustable.

12. A support device for an edematous scrotum comprising:
a body portion comprising a fabric belt adapted to be worn about a man's midsection, a first ring buckle for joining ends of the body portion together, and a plurality of second ring buckles disposed about an outer portion of the body portion;
a fabric receptacle adapted to receive and support an edematous scrotum comprising two support members rigidly fixed to the receptacle and each member adapted to couple with one of the ring buckles to thereby provide adjustable comforting support to the scrotum.

13. The device of claim 12, further comprising friction and/or pressure reducing pads on an inside surface of the belt adjacent the wearer's skin.

14. The device of claim 12, further comprising an insert in the receptacle for cushioning and/or cooling the scrotum.

15. The device of claim 14, wherein the insert is removable.

16. The device of claim 12, further comprising one or more pouches in the wall of the receptacle for receiving scrotum-cooling products.
